(19) United States
(12) Patent Application Publication
(10) Pub. No.: US 2021/0350917 A1
(43) Pub. Date: Nov. 11, 2021

(54) SYSTEM AND METHOD TO IMPROVE INTERACTION BETWEEN USERS THROUGH MONITORING OF EMOTIONAL STATE OF THE USERS AND REINFORCEMENT OF GOAL STATES

(71) Applicant: Steve CURTIS, Vancouver (CA)

(72) Inventor: Steve CURTIS, Vancouver (CA)

(21) Appl. No.: 17/278,539

(22) PCT Filed: Sep. 20, 2019
(86) PCT No.: PCT/CA2019/051340

§ 371 Date: Mar. 22, 2021

Related U.S. Application Data
(60) Provisional application No. 62/734,490, filed on Sep. 21, 2018.
(60) Provisional application No. 62/734,522, filed on Sep. 21, 2018.
(60) Provisional application No. 62/734,553, filed on Sep. 21, 2018.
(60) Provisional application No. 62/734,608, filed on Sep. 21, 2018.

(51) Int. Cl. G16H 40/63; G06F 1/16

(52) U.S. Cl. CPC G16H 40/63; G06F 1/163

(57) ABSTRACT

Disclosed is a system and method for monitoring interaction between a plurality of users for determining the emotional state of the users and modulating biorhythms of the users based on feedback. The method includes the step of collecting biorhythm data of the user through a wearable user device. The method includes the step of receiving the biorhythm data of the users through a computing device. The method includes the step of establishing an interaction with the users over the communication network through an artificial intelligence (AI) based agent module. The method includes the step of analyzing and displaying emotional data of the users in real-time through an emotional data displaying module. The method includes the step of modulating biorhythms of the users based on the feedback emitted from the computing device through a feedback module.

### TECHNICAL FIELD

The present invention relates to biofeedback, in particular to a system and method to improve the interaction between users through monitoring of the emotional state of the users and reinforcement of goal states.

### BACKGROUND ART

This specification recognizes that mainstream consumer solutions are limited in their ability to optimize communication between two or more people based on real-time physiological data. Furthermore, there are no solutions that coach recipients through various forms of feedback to improve communication outcomes. Improved communication outcomes may include better relationship and trust-building, better distribution and acquisition of information between parties, or greater enjoyment and satisfaction from the encounter. Systems that track physiological markers associated with a person's emotional and psychological state provide deeper insight into how a person is reacting to a real or simulated interpersonal encounter. Most importantly, these devices can show instantaneous changes in physiology which can be mapped to events as they occur.

Further, this specification recognizes that there are various problems or no solutions exist to accurately determine the emotional state of the user or establishing communication among the users. Physiological patterns in various bio-signals and biorhythms can be identified to be associated with specific stress states and emotions. Various systems and methods exist to empirically or passively assess the emotional state of the user based on the biological parameters. However, these systems have difficulty deriving insights from the limited data they process. These systems are not intelligent enough to monitor the conversational behavior of the users in different situations and retrieve the inference out of the conversation to understand the emotional state of the user.

Typically, the usage of words (speech or text) by one person carries different emotional weights than another person saying the same words. Likewise, the interpreter, the person on the receiving end of these words, will likely interpret the emotional intensity and the type of emotion differently. Thus it is difficult for any existing system to understand the actual emotion attached to the words used by the user. There is not a clear common denominator as to how emotion is perceived. Furthermore, since every person may have different emotions/feelings, any machine learning model or feedback/gesture capturing model that is taught emotions based on human reactions will be biased based on the users in the data-set.

Discussions are much more difficult when the communicating parties become emotional. The conversations may become emotional for a number of reasons. When making decisions in meetings or other life settings, people can go by assumptions about how the people “feel” about an issue. This may be described as a general feeling that one has about the outcome of a situation. But different people may think and feel differently in the same situation. Sometimes, it is hard to measure, quantify, or consistently, the shifting sentiment a person has towards various events or points in a conversation as it occurs in real-time. Two general goals of conversation are that it is a means to discover new information and second—to build a connection with another person. This can occur by spending time with a person and disclosing personal information.

Therefore there is a need for a system and method to provide a cognitive platform to monitor user interactions and to determine the emotional state of the user to provide assistance based on the emotional state of the user. Further, there is a need for an efficient system that captures user communication data in association with biorhythms and biodata to generate training datasets for a software learning agent. Furthermore, there is a need for a system and method that interacts with the users based on a plurality of biophysical states of the users determined such as Heart rate variability (HRV), Electroencephalography (EEG), etc. Also, there exists a need for systems and methods for helping people to communicate better with each other. Additionally, there is a need for a system and method which will stimulate a user, such as through a user's auditory system as a non-limiting example, while influencing biorhythms including emotional state of the user.

Thus, in view of the above, there is a long-felt need in the industry to address the aforementioned deficiencies and inadequacies.

Further limitations and disadvantages of conventional and traditional approaches will become apparent to one of skill in the art through comparison of described systems with some aspects of the present disclosure, as set forth in the remainder of the present application and with reference to the drawings.

### SUMMARY OF INVENTION

A system to monitor the interaction between a plurality of users to determine the emotional state of the users and modulate biorhythms of the users based on feedback over a communication network is provided substantially, as shown in and/or described in connection with at least one of the figures, as set forth more completely in the claims.

The present invention provides a method for monitoring interaction between a plurality of users for determining the emotional state of the users and modulating biorhythms of the users based on feedback over a communication network. The method includes the step of collecting biorhythm data of the user through a wearable user device configured to be worn on the user's body, near the body, or placed in the user's body (implantable). The method includes the step of receiving the biorhythm data of the users through a computing device communicatively connected with the wearable user device over a communication network. The method includes the step of establishing an interaction with the users over the communication network through an artificial intelligence (AI) based agent module. The method includes the step of analyzing and displaying emotional data of the users in real-time through an emotional data displaying module. The method includes the step of modulating biorhythms of the users based on the feedback emitted from the computing device through a feedback module.

The (AI) based agent module performs a plurality of steps that initiates with a step of receiving the biorhythm data from the wearable user device and monitor the interactions of a plurality of users and retrieves relevant data for analysis through a tracking module. The tracking module is integrated with one or more messaging platforms and one or more voice platforms of the computing device corresponding to the users to monitor textual interactions and audio interactions of the users. The tracking module processes the relevant data and the retrieved parameters to generate training data. The method includes the step of receiving and processing the training data to determine the emotional state of the user in a plurality of scenarios through a software learning agent module. The method includes the step of initiating the interaction with the user and assist the user based on the learned data received from the software learning agent module through a virtual chat-bot module. The method includes the step of facilitating the user to connect and interact with a plurality of other users through a community module. The community module facilitates the plurality of users to interact with each other and share emotional state and biorhythm data among the other users over the communication network. The method includes the step of allowing the user to access the emotion data of the other users through a sync module.

The emotional data displaying module performs a plurality of steps that initiates with a step of analyzing the biorhythm data and computing an emotional score of the user to generate one or more insights through an algorithmic module. The emotional score is indicative of the emotional state of the user during the interactions. The method includes the step of graphically representing a plurality of emotional cycles for a specific time duration for the user through a visualization module. The visualization module displays the insights and emotional scores of the users on the computing device associated with the users.

The feedback module performs a plurality of steps that initiates with a step of collecting physiological data of at least one physiological property of the user through a physiological data collection engine. The method includes the step of processing the physiological data into at least one biosignal through a biosignal generating engine. The method includes the step of monitoring and measuring the biosignal for a feedback activation condition through a feedback activation determining engine. The method includes the step of triggering feedback upon satisfying a feedback activation condition through feedback generating engine. The feedback activation condition triggers feedback when the measured value is more than one or more predetermined threshold values.

In an aspect, the tracking module retrieves a plurality of parameters of the users from the biorhythm data and monitoring data. The plurality of parameters includes the location of the user, biorhythm data of the user, personal and social behavior of the user, and environment, month, day, and time of the interactions.

In an aspect, the plurality of scenarios includes but not limited to contexts, situations, and environments. The software learning agent module is adaptable to continuously learn the contexts, situations, and environments based on the received training data and stores the learned data in a database.

In an aspect, the virtual chat-bot module interacts with the user to assist to improve the emotional state of the user.

In an aspect, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

Another aspect of the present invention relates to a system to monitor the interaction between a plurality of users to determine the emotional state of the users and modulate biorhythms of the users based on feedback over a communication network. The system includes a wearable user device and a computing unit. The wearable user device configured to be worn on the user's body, near the body or placed in the user's body (implantable) to collect biorhythm data of the user. The computing unit is communicatively connected with the wearable user device to receive the biorhythm data of the users over a communication network. The computing unit includes a processor, and a memory communicatively coupled to the processor. The memory includes an artificial intelligence (AI) based agent module, an emotional data displaying module, and a feedback module.

The artificial intelligence (AI) based agent module establishes an interaction with the users over the communication network. The emotional data displaying module analyzes and displays emotional data of the users in real-time. The feedback module configured with the wearable user device to modulate biorhythms of the users based on the feedback emitted from the computing device.

The (AI) based agent module includes a tracking module, a software learning agent module, a virtual chat-bot module, a community module, and a sync module. The tracking module receives the biorhythm data from the wearable user device and monitors the interactions of a plurality of users and retrieves relevant data for analysis. The tracking module is integrated with one or more messaging platforms and one or more voice platforms of the computing device corresponding to the users to monitor textual interactions and audio interactions of the users. The tracking module processes the relevant data and the retrieved parameters to generate training data. The relevant data is pertaining to text, sentiment, and audio and the tracking module performs text analysis, sentiment analysis, and signal processing on the audio. The software learning agent module receives and processes the training data to determine the emotional state of the user in a plurality of scenarios. The virtual chat-bot module initiates the interaction with the user and assists the user based on the learned data received from the software learning agent module. The community module facilitates the user to connect and interact with a plurality of other users. The community module facilitates the plurality of users to interact with each other and share emotional state and biorhythm data among the other users over the communication network. The sync module allows the user to access the emotion data of the other users.

The emotional data displaying module includes an algorithmic module and a visualization module. The algorithmic module analyzes the biorhythm data and computes an emotional score of the user to generate one or more insights. The emotional score is indicative of the emotional state of the user during the interactions. The visualization module graphically represents a plurality of emotional cycles for a specific time duration for the user. The visualization module displays the insights and emotional scores of the users on the computing device associated with the users.

The feedback module includes a physiological data collection engine, a biosignal generating engine, a feedback activation determining engine, and a feedback generating engine. The physiological data collection engine collects physiological data of at least one physiological property of the user. The biosignal generating engine processes the physiological data into at least one biosignal. The feedback activation determining engine monitors and measures the biosignal for a feedback activation condition. The feedback generating engine triggers feedback upon satisfying a feedback activation condition. The feedback activation condition triggers feedback when the measured value is more than one or more predetermined threshold values.

Accordingly, one advantage of the present invention is that it actively assists the users to improve their emotional and psychological state based on the data learned by the software learning agent module.

Accordingly, one advantage of the present invention is that it controls (increase or decrease) an involuntary or unconscious physiological process by self-regulating and exercising control over physiological variables.

Accordingly, one advantage of the present invention is that it provides a social platform to the users where they share their emotional data and allow other users to visualize the same to improve and work on their emotional state.

Accordingly, one advantage of the present invention is that it provides a ratio scale (with an absolute zero) that receives emotional data and displays it linearly.

Accordingly, one advantage of the present invention is that it provides the emotional data of the users periodically to help the users to optimize their emotional and psychological state over time and allowing them to feel more consistently in a positive state.

Other features of embodiments of the present invention will be apparent from accompanying drawings and from the detailed description that follows.

Yet other objects and advantages of the present invention will become readily apparent to those skilled in the art following the detailed description, wherein the preferred embodiments of the invention are shown and described, simply by way of illustration of the best mode contemplated herein for carrying out the invention. As we realized, the invention is capable of other and different embodiments, and its several details are capable of modifications in various obvious respects, all without departing from the invention. Accordingly, the drawings and description thereof are to be regarded as illustrative in nature, and not as restrictive.

### BRIEF DESCRIPTION OF DRAWINGS

In the figures, similar components and/or features may have the same reference label. Further, various components of the same type may be distinguished by following the reference label with a second label that distinguishes among the similar components. If only the first reference label is used in the specification, the description applies to any one of the similar components having the same first reference label irrespective of the second reference label.

FIG. 1

FIG. 1 illustrates a block diagram of the present system to monitor the interaction between a plurality of users to determine the emotional state of the users and modulate biorhythms of the users based on feedback over a communication network, in accordance with one embodiment of the present invention.

FIG. 2

FIG. 2 illustrates a network implementation of the present system, in accordance with one embodiment of the present invention.

FIG. 3

FIG. 3 illustrates a block diagram of the various modules within a memory of a computing device, in accordance with another embodiment of the present invention.

FIG. 4

FIG. 4 illustrates a flowchart of the method for monitoring interaction between a plurality of users for determining the emotional state of the users and modulating biorhythms of the users based on feedback over a communication network, in accordance with an alternative embodiment of the present invention.

FIG. 5

FIG. 5 illustrates a flowchart of the plurality of steps performed by an artificial intelligence (AI) based agent module, in accordance with an alternative embodiment of the present invention.

FIG. 6

FIG. 6 illustrates a flowchart of the plurality of steps performed by an emotional data displaying module, in accordance with an alternative embodiment of the present invention.

FIG. 7

FIG. 7 illustrates a flowchart of the plurality of steps performed by a feedback module, in accordance with an alternative embodiment of the present invention.

### DESCRIPTION OF EMBODIMENTS

The present disclosure is best understood with reference to the detailed figures and description set forth herein. Various embodiments have been discussed with reference to the figures. However, those skilled in the art will readily appreciate that the detailed descriptions provided herein with respect to the figures are merely for explanatory purposes, as the methods and systems may extend beyond the described embodiments. For instance, the teachings presented and the needs of a particular application may yield multiple alternative and suitable approaches to implement the functionality of any detail described herein. Therefore, any approach may extend beyond certain implementation choices in the following embodiments.

References to “one embodiment,” “at least one embodiment,” “an embodiment,” “one example,” “an example,” “for example,” and so on indicate that the embodiment(s) or example(s) may include a particular feature, structure, characteristic, property, element, or limitation but that not every embodiment or example necessarily includes that particular feature, structure, characteristic, property, element, or limitation. Further, repeated use of the phrase “in an embodiment” does not necessarily refer to the same embodiment.

Methods of the present invention may be implemented by performing or completing manually, automatically, or a combination thereof, selected steps or tasks. The term “method” refers to manners, means, techniques and procedures for accomplishing a given task including, but not limited to, those manners, means, techniques, and procedures either known to, or readily developed from known manners, means, techniques and procedures by practitioners of the art to which the invention belongs. The descriptions, examples, methods, and materials presented in the claims and the specification are not to be construed as limiting but rather as illustrative only. Those skilled in the art will envision many other possible variations within the scope of the technology described herein.

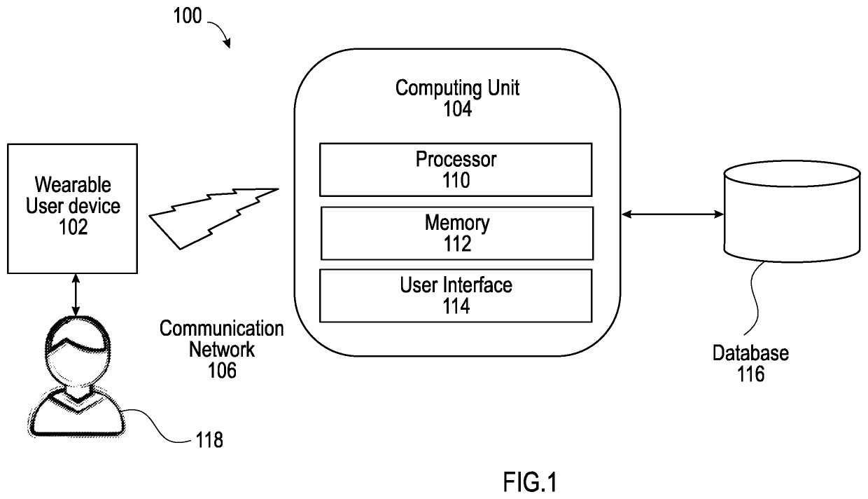

FIG. 1 illustrates a block diagram of the present system 100 to monitor the interaction between a plurality of users to determine the emotional state of the users and modulate biorhythms of the users based on feedback over a communication network, in accordance with one embodiment of the present invention. The system 100 includes a wearable user device 102, and a computing device 104. The wearable user device 102 is configured to be worn on the user's body, near the body, or placed in the user's body (implantable) to collect biorhythm data of the user 118. Examples of the wearable user device 102 include but not limited to the implantable, wireless sensor device, smartwatch, smart jewelry, fitness tracker, smart cloth, etc. In an embodiment, the wearable user device 102 includes various sensors to detect one or more parameters pertaining to the emotions of the user 118. In an embodiment, the wearable user device 102 may include a flexible body that can be secured around the user's body to collect the biorhythm data. In an embodiment, and the wearable user device 102 may including a securing mechanism to secure the wearable user device 102 may in a closed loop around a wrist of the user 118. Further, the wearable user device 102 may be any wearable such as an on-body sticker or 3d-printed device that is directly printed on the skin, or a device that placed on the body with an adhesive. The wearable user device 102 may utilize various wired or wireless communication protocols to establish communication with the computing unit 104.

The computing device 104 is communicatively connected with the wearable user device 102 to receive the biorhythm data of the users over a communication network 106. Communication network 106 may be a wired or a wireless network, and the examples may include but are not limited to the Internet, Wireless Local Area Network (WLAN), Wi-Fi, Long Term Evolution (LTE), Worldwide Interoperability for Microwave Access (WiMAX), General Packet Radio Service (GPRS), Bluetooth (BT) communication protocols, Transmission Control Protocol and Internet Protocol (TCP/IP), User Datagram Protocol (UDP), Hypertext Transfer Protocol (HTTP), File Transfer Protocol (FTP), ZigBee, EDGE, infrared (IR), Z-Wave, Thread, 5G, USB, serial, RS232, NFC, RFID, WAN, and/or IEEE 802.11, 802.16, 2G, 3G, 4G cellular communication protocols

Examples of the computing device 104 include but not limited to a laptop, a desktop, a smartphone, a smart device, a smartwatch, a phablet, and a tablet. The computing device 104 includes a processor 110, a memory 112 communicatively coupled to the processor 110, and a user interface 114. The computing device 104 is communicatively coupled with a database 114. The database 116 receives, stores, and process the emotional data and referral data which can be used for further analysis and prediction so that the present system can learn and improve the analysis by using the historical emotional data. Although the present subject matter is explained considering that the present system 100 is implemented on a cloud device, it may be understood that the present system 100 may also be implemented in a variety of computing systems, such as an Amazon elastic compute cloud (Amazon EC2), a network server, and the like. The data collected from the user is constantly being monitored and sent to the server (when convenient and connected), where it is stored, analyzed, and modeled. New AI models are generated on the server and then downloaded to the computing devices at various intervals.

Processor 110 may include at least one data processor for executing program components for executing user- or system-generated requests. A user may include a person, a person using a device such as those included in this invention, or such a device itself. Processor 110 may include specialized processing units such as integrated system (bus) controllers, memory management control units, floating point units, graphics processing units, digital signal processing units, etc.

Processor 110 may include a microprocessor, such as AMD® ATHLON® microprocessor, DURON® microprocessor OR OPTERON® microprocessor, ARM's application, embedded or secure processors, IBM® POWERPC®, INTEL'S CORE® processor, ITANIUM® processor, XEON® processor, CELERON® processor or other line of processors, etc. Processor 110 may be implemented using mainframe, distributed processor, multi-core, parallel, grid, or other architectures. Some embodiments may utilize embedded technologies like application-specific integrated circuits (ASICs), digital signal processors (DSPs), Field Programmable Gate Arrays (FPGAs), etc.

Processor 110 may be disposed of in communication with one or more input/output (I/O) devices via an I/O interface. I/O interface may employ communication protocols/methods such as, without limitation, audio, analog, digital, RCA, stereo, IEEE-1394, serial bus, universal serial bus (USB), infrared, PS/2, BNC, coaxial, component, composite, digital visual interface (DVI), high-definition multimedia interface (HDMI), RF antennas, S-Video, VGA, IEEE 802.n/b/g/n/x, Bluetooth, cellular (e.g., code-division multiple access (CDMA), high-speed packet access (HSPA+), global system for mobile communications (GSM), long-term evolution (LTE), WiMax, or the like), etc.

Memory 112, which may be a non-volatile memory or a volatile memory. Examples of non-volatile memory may include, but are not limited to flash memory, a Read Only Memory (ROM), a Programmable ROM (PROM), Erasable PROM (EPROM), and Electrically EPROM

(EEPROM) memory. Examples of volatile memory may include but are not limited Dynamic Random Access Memory (DRAM), and Static Random-Access memory (SRAM).

The user interface 114 may present the monitored interaction data, determine emotional data and modulated biorhythms data as per the request of an administrator of the present system. In an embodiment, the user interface (UI or GUI) 114 is a convenient interface for accessing the platform and viewing the products or services. The biorhythmic data includes but not limited to heart rate, heart rate variability, electrodermal activity (EDA)/Galvanic skin response (GSR), breathing rate, 3D accelerometer data, and gyroscope data, body temperature, among others. The biorhythmic data can be processed to generate the signals based on mathematical description or algorithms. The algorithms may be introduced via software. There is potential that data is processed on the wearable user device end. Data may also be stored there temporarily before acted upon.

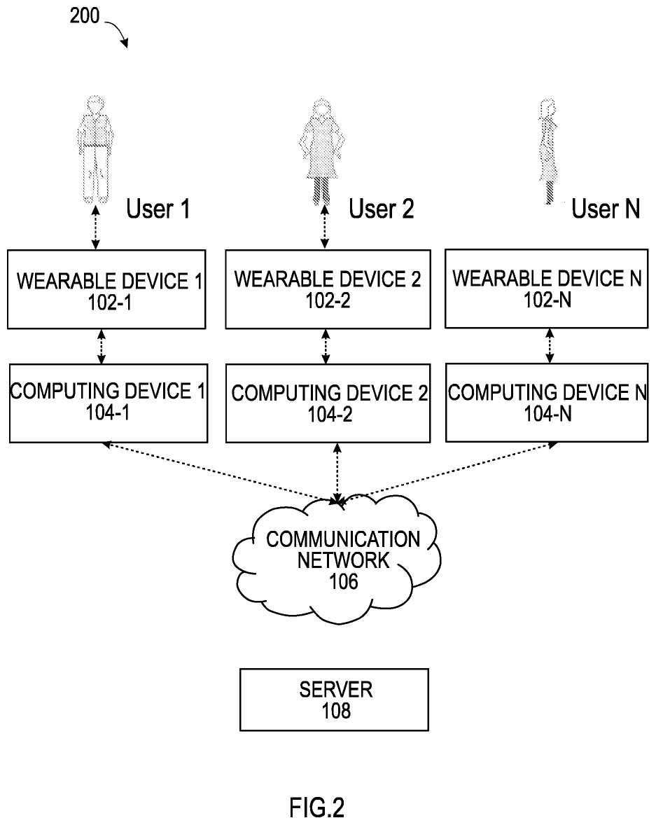

FIG. 2 illustrates a network implementation 200 of the present system, in accordance with one embodiment of the present invention. FIG. 2 is explained in conjunction with FIG. 1. The computing devices 104-1, 104-2, and 104-N are communicatively connected with the wearable user devices 102-1, 102-2, and 102-N to receive the biorhythm data of the users over the communication network 106. A server 108 stores and processes the monitored interaction data, determine emotional data and modulated biorhythms data. The computing device 104 or wearable user device 102 may initiate a sound notification (any type of sound). Based on the user's current emotional state score, different sounds should be issued by one or more of the wearable user devices 102 to inform the users to do one of several different behaviors. It may be appreciated that behavior may not be limited to one behavior, and sound could signal a plurality (multiple) of actions. The behavior associated with the sound should help the user change their behavior to move closer to the user's desired/preset emotional state, or move towards changing a more specific biorhythm.

In an aspect, the network architecture of the wearable user device 102 and the computing device 104 can include one or more Internet of Things (IoT) devices. In a typical network architecture of the present disclosure can include a plurality of network devices such as transmitter, receivers, and/or transceivers that may include one or more IoT devices.

In an embodiment, the wearable user device, 102 can directly interact with the cloud and/or cloud servers and IoT devices. The IoT devices are utilized to communicate with many wearable user devices or other electronic devices. The IoT devices may provide various feedback through sensing or controlling mechanism to collect interaction between the users and communicate the emotional state of the users. The data and/or information collected can be directly stored in the cloud server without taking any space on the user mobile and/or portable computing device. The mobile and/or portable computing device can directly interact with a server and receive information for feedback activation to trigger deliver the feedback. Examples of the feedback include but not limited to auditory feedback, haptic feedback, tactile feedback, vibration feedback, or visual feedback from a primary wearable device, a secondary wearable device, a separate computing device (i.e. mobile), or IoT device (which may or may not be a computing device).

As used herein, the IoT devices can be a device that includes sensing and/or control functionality as well as a WiFi™ transceiver radio or interface, a Bluetooth™ transceiver radio or interface, a Zigbee™ transceiver radio or interface, an Ultra-Wideband (UWB) transceiver radio or interface, a WiFi-Direct transceiver radio or interface, a Bluetooth™ Low Energy (BLE) transceiver radio or interface, and/or any other wireless network transceiver radio or interface that allows the IoT device to communicate with a wide area network and with one or more other devices. In some embodiments, an IoT device does not include a cellular network transceiver radio or interface, and thus may not be configured to directly communicate with a cellular network. In some embodiments, an IoT device may include a cellular transceiver radio and may be configured to communicate with a cellular network using the cellular network transceiver radio.

A user may communicate with the network devices using an access device that may include any human-to-machine interface with network connection capability that allows access to a network. For example, the access device may include a stand-alone interface (e.g., a cellular telephone, a smartphone, a home computer, a laptop computer, a tablet, a personal digital assistant (PDA), a computing device, a wearable device such as a smartwatch, a wall panel, a keypad, or the like), an interface that is built into an appliance or other device e.g., a television, a refrigerator, a security system, a game console, a browser, or the like), a speech or gesture interface (e.g., a Kinect™ sensor, a Wiimote™, or the like), an IoT device interface (e.g., an Internet-enabled devices such as a wall switch, a control interface, or other suitable interface), or the like. In some embodiments, the access device may include a cellular or other broadband network transceiver radio or interface and may be configured to communicate with a cellular or other broadband network using the cellular or broadband network transceiver radio. In some embodiments, the access device may not include a cellular network transceiver radio or interface.

In an embodiment, the users may be provided with an input/display screen which is configured to display information to the user about the current status of the system. The input/display screen may take input from an input apparatus, in the current example buttons. The input/display screen may also be configured as a touch screen or may accept input for determining vitals or bio-signals through touch or haptic based input system. The input buttons and/or screen are configured to allow a user to respond to input prompt from the system regarding needed user input.

The information which may be displayed on the screen to the user may be, for instance, the number of treatments provided, bio-signals values, vitals, the battery charge level, and volume level. The input/display screen may take information from a processor which may also be used as the waveform generator or may be a separate processor. The processor provides available information for display to the user allowing the user to initiate menu selections. The input/display screen may be a liquid crystal display to minimize power drain on the battery. The input/display screen and the input buttons may be illuminated to provide a user with the capability to operate the system in low light levels. Information can be obtained from a user through the use of the input/display screen.

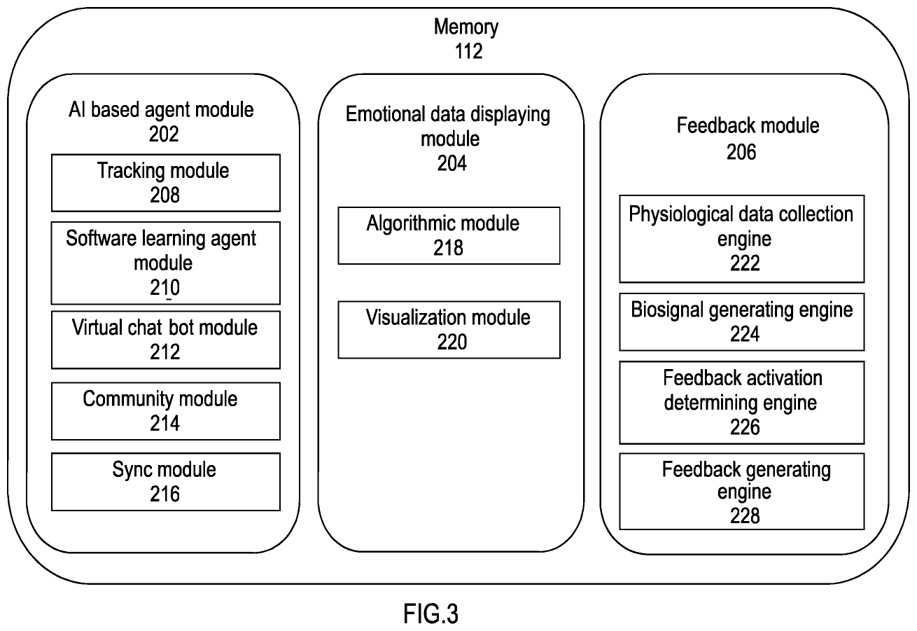

FIG. 3 illustrates a block diagram of the various modules within a memory 112 of a computing device 104, in accordance with another embodiment of the present invention. FIG. 3 is explained in conjunction with FIG. 1. The memory 110 includes an artificial intelligence (AI) based agent module 202, an emotional data displaying module 204, and a feedback module 206.

The artificial intelligence (AI) based agent module 202 establishes an interaction with the users over the communication network. The emotional data displaying module 204 analyzes and displays emotional data of the users in real-time. The feedback module 206 configured with the wearable user device to modulate biorhythms of the users based on the feedback emitted from the computing device.

The (AI) based agent module 202 includes a tracking module 208, a software learning agent module 210, a virtual chat-bot module 212, a community module 214, and a sync module 216. The tracking module 208 receives the biorhythm data from the wearable user device and monitors the interactions of a plurality of users and retrieves relevant data for analysis. The tracking module 208 is integrated with one or more messaging platforms and one or more voice platforms of the computing device corresponding to the users to monitor textual interactions and audio interactions of the users. The tracking module 208 processes the relevant data and the retrieved parameters to generate training data. In an embodiment, the tracking module 208 retrieves a plurality of parameters of the users from the biorhythm data and monitored data. The plurality of parameters includes the location of the user, biorhythm data of the user, personal and social behavior of the user, and environment, month, day, and time of the interactions. In an embodiment, the plurality of scenarios includes but not limited to contexts, situations, and environments.

The software learning agent module 210 receives and processes the training data to determine the emotional state of the user in a plurality of scenarios. In an embodiment, the biorhythm data, emotional data, relevant data, and training data can be combined or deconstructed or converted in various ways to aid modeling. The training data can be utilized to train the various algorithms used to achieve the objective of the present system. The training data includes input data and the corresponding expected output. Based on the training data, the algorithm can learn how to apply various mechanisms such as neural networks, to learn, produce, and predict the emotional state of the user in the plurality of scenarios, so that it can accurately determine the emotional state when later presented with new input data.

The software learning agent module 210 is adaptable to continuously learn the contexts, situations, and environments based on the received training data and stores the learned data in a database. The virtual chat-bot module 212 initiates the interaction with the user and assist the user based on the learned data received from the software learning agent module. In an embodiment, the virtual chat-bot module 212 interacts with the user to assist to improve the emotional state of the user.

The community module 214 facilitates the user to connect and interact with a plurality of other users. The community module 214 facilitates the plurality of users to interact with each other and share emotional state and biorhythm data among the other users over the communication network. The community module 214 enables the user to view a list of existing friends and further enables the user to search for other users via a text-based name search. The users can also send friend requests to other users. The other users receive a notification on receiving the friend request from the users. The users can accept or decline the friend request. The community module 214 further allows both the users to access the general statistics related to the emotional state of each other. Additionally, the user can interact with each other through a messaging module integrated within the community module 214.

The sync module 216 allows the user to access the emotion data of the other users. The sync module 216 utilizes an initiation and acceptance protocols to enables the user to accept/decline the friend request and allow/disallow the other users to access his/her emotional data. Alternatively, the users may turn on a setting that is (bidirectional or unidirectional) to allow both the users to receive expanded access to the one or each other's data. Regardless of the protocol, and directionality of the sync, the end benefit is that the other person's psychological state or emotional state score should be visualized with options to view past periods of time. Most importantly, assuming real-time data is streaming from each other's devices to their secondary devices (mobile phones), the users should be able to view each other's real-time emotional scores. These emotional scores can be divided into zones that can be linearly divided or along zones in a 2-axis array, or in zones based on the n-dimensional matrix. Overall, the zones follow some clear gradient that is communicated to users in various places in the product. The syncing states between two parties also allow evaluations to be made and insights to be derived between the 2 or more synced accounts.

In an additional embodiment, the present invention uses a multi-syncing module. The multi-syncing module enables more than two user accounts to sync up (get connected with others) to visualize the emotional data of each other. The use of location-based services facilitates easy recognition when multi-syncing can occur. If multiple devices are detected on the software application or if the GPS services detect that computing units are within a short distance of each other, then those users—who have already acknowledged each other as friends on the community module—will appear most prominent on the list.

The multi-syncing module provides advanced insights and shows many groups statistics. The notifications in the multi-syncing module may include changes in groups results. In an embodiment, the sync factor can be turned off at any given time by anyone. In the multi-syncing module, if one user turns off their sync feature, other members who are still synced up will remain connected. The secondary computing units (not shown in FIG.) that display related sync results may offer visual, auditory, or haptic/tactile feedback that progressively synchronizes various behaviors such as breathing rate and aspect of the breathing cycle (whether both people are at the peak of inhalation or trough of exhalation). Further, the sync feature encompasses any combinations of biorhythms including brain waves such as EEG.

In an embodiment, the software application identifies the target points on bio-signals, or users can mutually or individually select goals/targets points for biorhythm measurements. Once these targets are identified, the feedback of various types will then work change behavior and biorhythms to move them closer to this target point. The target can be static or dynamic. The objective of the syncing is to move the emotional states of the two or more users closer together, but only in a positive direction. Moving one user who is in a negative emotional state to closer alignment with a person in a positive emotional state will yield a more positive conversational experience between the two users.

In an embodiment, the sync module 216 comprises a recording module to record the conversation. The recording module acts as a virtual button over an interface that allows the user to turn ON/OFF the recording. Audio is then recorded through the microphone of a secondary computing unit if there is one or a similar tool available. The sync module 216 comprises a language processing module that applies to the recorded audio files to transform the dialogue audio waves into the transcribed language. The transcribed language is further processed based on sentiment and content and matched temporally with biorhythms of the speaker's emotional scores.

In an embodiment, a visualized display of the emotional scores of the users in the meeting is displayed in real-time on the interfaces of the secondary computing units of all the users. Notifications can be sent to one or more users (either visual—i.e., textual or graphics), auditory (a short audio clip) or haptic (either via the wearable device or secondary computing unit). These notifications can be sent when there are marked biorhythm changes that occur in either participant/user.

The emotional data displaying module 204 includes an algorithmic module 218, and a visualization module 220. The algorithmic module 218 analyzes the biorhythm data and computes an emotional score of the user to generate one or more insights. The emotional score is indicative of the emotional state of the user during the interactions. The visualization module 220 graphically represents a plurality of emotional cycles for a specific time duration for the user. The visualization module 220 displays the insights and emotional scores of the users on the computing device associated with the users. In an embodiment, the visualization module 220 displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

The feedback module 206 includes a physiological data collection engine 222, a biosignal generating engine 224, a feedback activation determining engine 226, and feedback generating engine 228. The physiological data collection engine 222 collects physiological data of at least one physiological property of the user. The biosignal generating engine 224 processes the physiological data into at least one biosignal. The feedback activation determining engine monitors and measures the biosignal for a feedback activation condition. The feedback generating engine 228 triggers feedback upon satisfying a feedback activation condition. The feedback activation condition triggers feedback when the measured value is more than one or more predetermined threshold values.

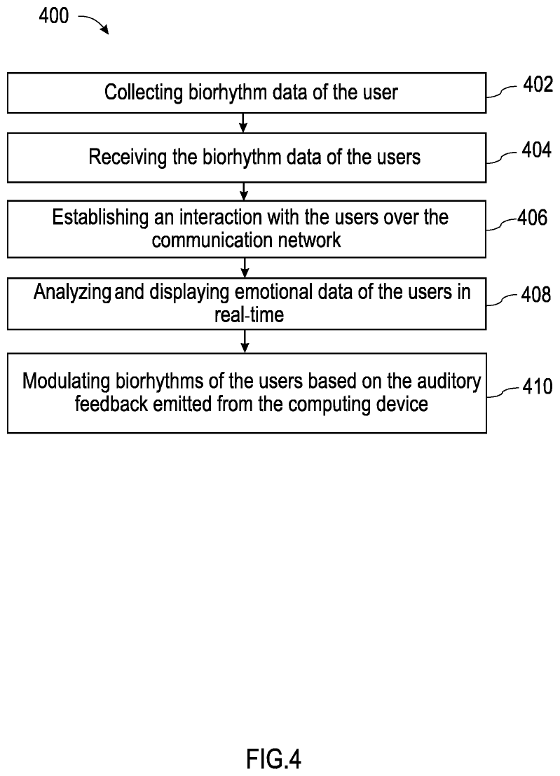

FIG. 4 illustrates a flowchart 400 of the method for monitoring interaction between a plurality of users for determining the emotional state of the users and modulating biorhythms of the users based on feedback over a communication network, in accordance with an alternative embodiment of the present invention. The method includes step 402 of collecting biorhythm data of the user through a wearable user device configured to be worn on the user's body, near the body, or placed in the user's body (implantable). The method includes the step 404 of receiving the biorhythm data of the users through a computing device communicatively connected with the wearable user device over a communication network. The method includes the step 406 of establishing an interaction with the users over the communication network through an artificial intelligence (AI) based agent module. The method includes the step 408 of analyzing and displaying emotional data of the users in real-time through an emotional data displaying module. The method includes step 410 of modulating biorhythms of the users based on the feedback emitted from the computing device through a feedback module.

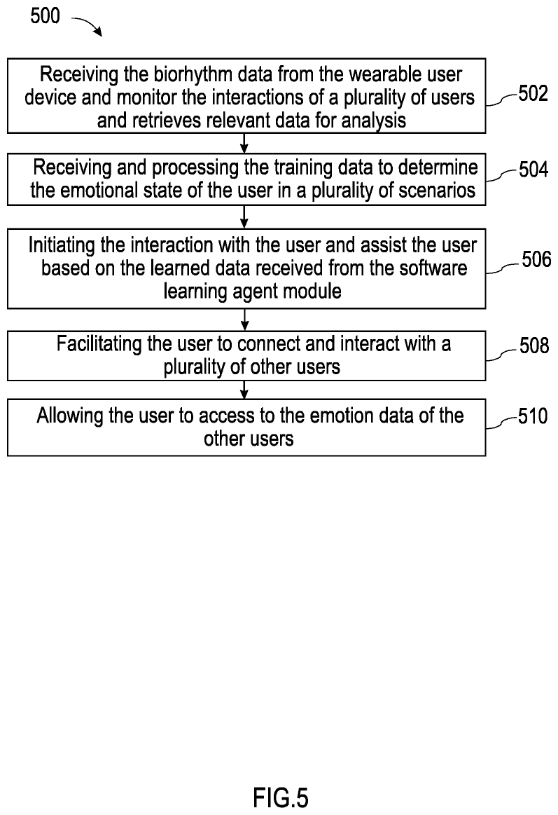

FIG. 5 illustrates a flowchart 500 of the plurality of steps performed by an artificial intelligence (AI) based agent module, in accordance with an alternative embodiment of the present invention. The (AI) based agent module performs a plurality of steps that initiates with a step 502 of receiving the biorhythm data from the wearable user device and monitor the interactions of a plurality of users and retrieves relevant data for analysis through a tracking module. The tracking module is integrated with one or more messaging platforms and one or more voice platforms of the computing device corresponding to the users to monitor textual interactions and audio interactions of the users. The tracking module processes the relevant data and the retrieved parameters to generate training data. In an embodiment, the tracking module retrieves a plurality of parameters of the users from the biorhythm data and monitored data. The plurality of parameters includes the location of the user, biorhythm data of the user, personal and social behavior of the user, and environment, month, day, and time of the interactions.

The method includes the step 504 of receiving and processing the training data to determine the emotional state of the user in a plurality of scenarios through a software learning agent module. In an embodiment, the plurality of scenarios includes but not limited to contexts, situations, and environments. The software learning agent module is adaptable to continuously learn the contexts, situations, and environments based on the received training data and stores the learned data in a database. The method includes the step 506 of initiating the interaction with the user and assist the user based on the learned data received from the software learning agent module through a virtual chat-bot module. In an embodiment, the virtual chat-bot module interacts with the user to assist to improve the emotional state of the user. The method includes the step 508 of facilitating the user to connect and interact with a plurality of other users through a community module. The community module facilitates the plurality of users to interact with each other and share emotional state and biorhythm data among the other users over the communication network. The method includes the step 510 of allowing the user to access the emotion data of the other users through a sync module.

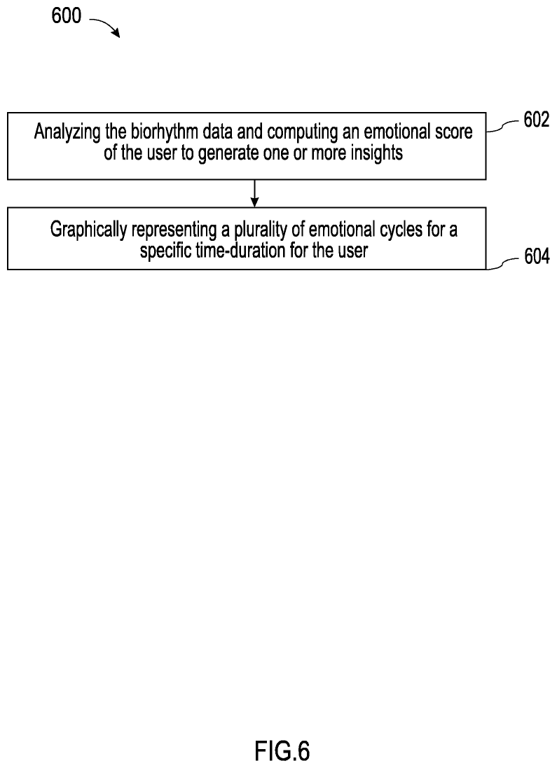

FIG. 6 illustrates a flowchart 600 of the plurality of steps performed by an emotional data displaying module, in accordance with an alternative embodiment of the present invention. The emotional data displaying module performs a plurality of steps that initiates with a step 602 of analyzing the biorhythm data and computing an emotional score of the user to generate one or more insights through an algorithmic module. The emotional score is indicative of the emotional state of the user during the interactions. The method includes step 604 of graphically representing a plurality of emotional cycles for a specific time duration for the user through a visualization module. The visualization module displays the insights and emotional scores of the users on the computing device associated with the users. In an embodiment, the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols which include colors or moving shapes.

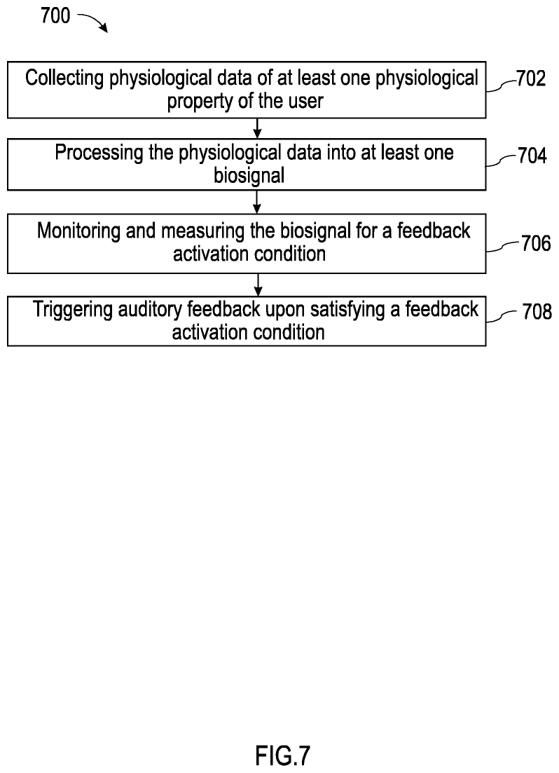

FIG. 7 illustrates a flowchart 700 of the plurality of steps performed by a feedback module, in accordance with an alternative embodiment of the present invention. The feedback module performs a plurality of steps that initiates with a step 702 of collecting physiological data of at least one physiological property of the user through a physiological data collection engine. The method includes the step 704 of processing the physiological data into at least one biosignal through a biosignal generating engine. The method includes the step 706 of monitoring and measuring the biosignal for a feedback activation condition through a feedback activation determining engine. The method includes the step 708 of triggering feedback upon satisfying a feedback activation condition through feedback generating engine. The feedback activation condition triggers feedback when the measured value is more than one or more predetermined threshold values.

Thus the present invention provides a cognitive platform to monitor user interactions and to determine the emotional state of the user to provide assistance based on the emotional state of the user. Further, the present invention captures the user communication data related to the biorhythm and bio-data of the users to generate training datasets for the software learning agent module. Furthermore, the present invention interacts with the users based on a plurality of biophysical states of the users determined such as Heart rate variability (HRV), Electroencephalography (EEG), etc. Further, the present invention provides a ratio scale that receives emotional data and visualizes it linearly. Furthermore, the present invention provides the emotional data of the users periodically to help the users to optimize their emotional and psychological state over time. Additionally, the present invention enables the users to draw personal insights based on their emotional data or the emotional data of the other users.

While embodiments of the present invention have been illustrated and described, it will be clear that the invention is not limited to these embodiments only. Numerous modifications, changes, variations, substitutions, and equivalents will be apparent to those skilled in the art, without departing from the scope of the invention, as described in the claims.

1. A system to monitor interaction between a plurality of users to determine emotional state of the users and modulate biorhythms of the users based on feedback over a communication network, the system comprising:
a wearable user device configured to collect biorhythm data of the user; and
a computing device is communicatively connected with the wearable user device to receive the biorhythm data of the users over the communication network, wherein the computing device comprising:
a processor; and
a memory communicatively coupled to the processor, wherein the memory stores instructions executed by the processor, wherein the memory comprising:
an artificial intelligence (AI) based agent module to establish an interaction with the users over the communication network, wherein the AI-based agent module comprising:
a tracking module to receive the biorhythm data from the wearable user device and monitor the interactions of a plurality of users and retrieves relevant data for analysis, wherein the tracking module is integrated with one or more messaging platforms and one or more voice platforms of the computing device corresponding to the users to monitor textual interactions and audio interactions of the users, wherein the tracking module processes the relevant data and the retrieved parameters to generate training data, wherein the relevant data is pertaining to text, sentiment, and audio and the tracking module performs text analysis, sentiment analysis, and signal processing on the audio;
a software learning agent module to receive and process the training data to determine the emotional state of the user in a plurality of scenarios;
a virtual chat-bot module to initiate the interaction with the user and assist the user based on the learned data received from the software learning agent module;
a community module to facilitate the user to connect and interact with a plurality of other users, wherein the community module facilitates the plurality of users to interact with each other and share emotional state and biorhythm data among the other users over the communication network; and
a sync module to allow the user to access to the emotion data of the other users;
an emotional data displaying module to analyze and display emotional data of the users in real-time, wherein the emotional data displaying module comprising:
an algorithmic module to analyze the biorhythm data and compute an emotional score of the user to generate one or more insights, wherein the emotional score is indicative of the emotional state of the user during the interactions; and
a visualization module to graphically represent a plurality of emotional cycles for a specific time duration for the user, wherein the visualization module displays the insights and emotional scores of the users on the computing device associated with the users; and
a feedback module configured with the wearable user device to modulate biorhythms of the users based on the feedback emitted from the computing device, wherein the feedback module comprising:
a physiological data collection engine to collect physiological data of at least one physiological property of the user;
a biosignal generating engine to process the physiological data into at least one biosignal;
a feedback activation determining engine to monitor and measure the biosignal for a feedback activation condition; and
a feedback generating engine to trigger feedback upon satisfying a feedback activation condition, wherein the feedback activation condition triggers the feedback when the measured value is more than one or more predetermined threshold values.

2. The system according to claim 1, wherein the tracking module retrieves a plurality of parameters of the users from the biorhythm data and monitored data, wherein the plurality of parameters comprising location of the user, biorhythm data of the user, personal and social behavior of the user, and environment, month, day, and time of the interactions.

3. The system according to claim 1, wherein the plurality of scenarios comprising contexts, situations, and environments, wherein the software learning agent module is adaptable to continuously learn the contexts, situations, and environments based on the received training data and stores the learned data in a database.

4. The system according to claim 1, wherein the virtual chat-bot module interacts with the user to assist to improve the emotional state of the user.

5. The system according to claim 1, wherein the visualization module displays emotional data in a plurality of manners on at least one of a two dimensional (2D) graph, and a three dimensional (3D) graphs by using at least one of a plurality of alpha-numeric characters, a plurality of geometric shapes, a plurality of holograms, and a plurality of symbols.

6. A method for monitoring interaction between a plurality of users for determining emotional state of the users and modulating biorhythms of the users based on feedback over a communication network, the method comprising steps of:
collecting biorhythm data of the user through a wearable user device;
receiving the biorhythm data of the users through a computing device communicatively connected with the wearable user device over the communication network;
establishing an interaction with the users over the communication network through an artificial intelligence (AI) based agent module, wherein the AI-based agent module performs a plurality of steps comprising:
receiving the biorhythm data from the wearable user device and monitor the interactions of a plurality of users and retrieves relevant data for analysis through a tracking module, wherein the tracking module is integrated with one or more messaging platforms and one or more voice platforms of the computing device corresponding to the users to monitor textual interactions and audio interactions of the users, wherein the tracking module processes the relevant data and the retrieved parameters to generate training data, wherein the relevant data is pertaining to text, sentiment, and audio and the tracking module performs text analysis, sentiment analysis, and signal processing on the audio;
receiving and processing the training data to determine the emotional state of the user in a plurality of scenarios through a software learning agent module;
initiating the interaction with the user and assist the user based on the learned data received from the software learning agent module through a virtual chat-bot module;
facilitating the user to connect and interact with a plurality of other users through a community module, wherein the community module facilitates the plurality of users to interact with each other and share emotional state and biorhythm data among the other users over the communication network; and
allowing the user to access the emotion data of the other users through a sync module;
analyzing and displaying emotional data of the users in real-time through an emotional data displaying module, wherein the emotional data displaying module performs a plurality of steps comprising:
analyzing the biorhythm data and computing an emotional score of the user to generate one or more insights through an algorithmic module, wherein the emotional score is indicative of the emotional state of the user during the interactions; and
graphically representing a plurality of emotional cycles for a specific time duration for the user through a visualization module, wherein the visualization module displays the insights and emotional scores of the users on the computing device associated with the users; and
modulating biorhythms of the users based on the feedback emitted from the computing device through a feedback module, wherein the feedback module performs a plurality of steps comprising:
collecting physiological data of at least one physiological property of the user through a physiological data collection engine;
processing the physiological data into at least one biosignal through a biosignal generating engine;
monitoring and measuring the biosignal for a feedback activation condition through a feedback activation determining engine; and
triggering feedback upon satisfying a feedback activation condition through a feedback generating engine, wherein the feedback activation condition triggers the feedback when the measured value is more than one or more predetermined threshold values.

7. The method according to claim 6, wherein the tracking module retrieves a plurality of parameters of the users from the biorhythm data and monitored data, wherein the plurality of parameters comprising location of the user, biorhythm data of the user, personal and social behavior of the user, and environment, month, day, and time of the interactions.

8. The method according to claim 6, wherein the plurality of scenarios comprising contexts, situations, and environments, wherein the software learning agent module is adaptable to continuously learn the contexts, situations, and environments based on the received training data and stores the learned data in a database.

9. The method according to claim 6, wherein the virtual chat-bot module interacts with the user to assist to improve the emotional state of the user.

10. The method according to claim 6, wherein the visualization module displays emotional data on a two dimensional (2D) graph, and a three dimensional (3D) graphs, wherein the visualization module displays a plurality of alpha-numeric characters, a plurality of geometric shapes, a plurality of holograms, and a plurality of symbols.
